(19) United States
(12) Patent Application Publication
(10) Pub. No.: US 2023/0130689 A1
(43) Pub. Date: Apr. 27, 2023

(54) IN VITRO DIAGNOSTIC SAMPLE TUBES AND METHODS OF USE THEREOF

(71) Applicants: Frank Allen Cable, JR., Media, PA (US); Orion Franceso CABLE, Amherst, MA (US); Sotirios GERAGONIS, Oxford, MA (US)

(72) Inventors: Frank Allen Cable, JR., Media, PA (US); Orion Franceso CABLE, Amherst, MA (US); Sotirios GERAGONIS, Oxford, MA (US)

(21) Appl. No.: 17/655,044

(22) Filed: Mar. 16, 2022

Related U.S. Application Data
(60) Provisional application No. 63/262,990, filed on Oct. 25, 2021.

(51) Int. Cl. B01L 3/00; A61B 10/00

(52) U.S. Cl. CPC B01L 3/50825; A61B 10/0096; B01L 2300/043

(57) ABSTRACT

A diagnostic sample tube, comprising a tube body having a tube cavity to receive a test sample; and a tube body lid; the tube body and the tube body lid are formed as a single piece; a mechanical fastening mechanism formed with the single piece, the mechanical fastening mechanism comprising an engagement protrusion and an engagement protrusion receiver; wherein, when the mechanical fastening mechanism is fastened, the mechanical fastening mechanism holds the tube body and the tube body lid in a closed position relative to one another with the cavity closed; and wherein, when the mechanical fastening mechanism is fastened, the engagement protrusion receiver surrounds a longitudinal length of the engagement protrusion.

### CROSS-REFERENCE TO RELATED APPLICATIONS

This U.S. non-provisional patent application claims the benefit of the filing date of U.S. Provisional Pat. Application serial no. 63/262,990, filed Oct. 25, 2021, the entire disclosure of which is incorporated herein by reference.

### FIELD

The present disclosure relates to in vitro diagnostic test sample tubes and methods of use thereof.

### BACKGROUND

Current In Vitro Diagnostic (IVD) methods for analysis of pathogens and other items of interest utilize amplification of target materials, e.g. genes or other biopolymers, as a means of generating a Concentration Threshold (Ct) value, as calculated by a diagnostic instrument.

A well-established nucleic acid amplification method termed loop-mediated isothermal amplification (LAMP) was first developed by Notomi et al. in 2007.

A second method, Polymerase Chain Reaction (PCR) is a technique for amplifying deoxyribonucleic acid (DNA) that dramatically boosted the pace of genetic research. In a matter of a few hours, PCR can make billions of copies of a specific segment of DNA. The 1993 Nobel Prize in Chemistry was given to Kary B. Mullis for PCR process.

Both of the above methods, as well as other IVD technologies, utilize a sample tube for containment of an analyte. The sample tube is typically injection-molded, and formed of a material such as polypropylene or polyethylene. The material is light transmissive, as to enable light transmittance into the sample tube, as well as allowing for the reflected light to be measured, which is typically the method by which the Ct value is obtained by the instrumentation.

A typical clinical workflow may involve capture of a host (patient) sample, via a swab introduced into either the nostril (nasal swab) or mouth (mouth swab) of the host; however, samples may also be obtained via the host’s blood or urine, depending upon the assay design. Oftentimes the initial sample may then be captured within a transport tube. Saliva collection oftentimes utilizes a collection funnel in conjunction with a transport tube.

Once the initial sample is captured, a lysing solution or buffer may be introduced and mixed with the host sample, after which a prescribed volume is introduced into the sample tube.

Reagents are also required to drive the amplification process. These reagents can either be freeze dried, i.e. lyophilized and loaded into the sample tube prior to use, or added at the time of test preparation in liquid or pellet form.

If the reagents are first lyophilized and added to the sample tube as part of the test kit manufacturing process, the lid feature is utilized to contain the freeze-dried slug of reagents, which may be referred to as a lyosphere, during transport.

Of importance, the lid must retain the lyosphere during transport, while also allowing for access when the analyte fluid mixture is added to the sample tube.

In support of the above requirement, currently available sample tubes most typically have a lid that can be snapped over the tube opening. The lid is typically attached to the tube body. Additionally, an annular ring is typically incorporated to maintain the lid position during transport to retain the lyosphere, while still allowing for ready access to the contents, by opening with only finger pressure, when the analyte is added to the tube.

Once the lid is closed, the sample tube may be agitated either by hand or via some mechanical means after which the tube is inserted into the instrument for initiation of the IVD test cycle.

The PCR test cycle involves repeated thermal cycling in order to amplify the target biopolymers. LAMP assays typically reach a set temperature and then maintain an isothermal environment throughout the test.

The required energy to heat the tube contents is typically transferred across the sample tube wall thickness via conduction, via a heater block, light absorption via LED or other light generating components or convection of either air or like fluids.

Since test throughput within a lab or clinician’s office is a key economic consideration, there is a focus on minimizing the time-to-results for any commercially viable IVD test. Additionally, by minimizing time-to-results infection control measures become more effective, by allowing for fast identification and quarantine of infected individuals within a given population.

To date, the above testing has historically occurred within a clinical setting as regulated by the U.S. Food and Drug Administration (FDA) Clinical Laboratory Improvement Amendments (CLIA). Tests are typically classified as moderate or high complexity and approved for CLIA labs only, due to the complexity of the test protocol, coupled with the potential risk of operator exposure to the analyte, such as the SARS-CoV-2 virus, Flu virus, HIV, MERS or other pathogens.

Due to the historic demand placed on CLIA labs by the Covid-19 pandemic, the U.S. Department of Health and Human Services (HHS) and FDA have placed a high priority on the development of next-generation Point of Care (PoC) tests for the SARS-Cov-2 virus. As a result, manufacturers are developing a variety of tests, including amplification tests (PCR, LAMP) set for sale and distribution within schools and business, as well as ultimately Over the Counter (OtC) sales to lay people for home use.

The design of current sample tubes may pose an unacceptable risk to the untrained user of PoC or OtC tests, since such user may not fully understand the risk of exposure to the contained analyte during the test procedure. Once amplified, the contained genetic material may pose an even higher risk to the test operator or nearby individuals, if the sample tube is opened either during or after the test cycle.

### SUMMARY

The present disclosure provides, among other things, in vitro diagnostic sample tubes which provided increased safety considerations for a user of a PoC or OtC test.

In one embodiment, a sample tube is provided which has a “safety lid” for a one-time closure event, after which the lid is mechanically locked in a closed position and provides increased tamper resistance relative to tubes of the current state of the art. Such a tube is suitable for a test protocol where all materials (e.g. sample, buffer, lysing agent and reagents) are added by the user to the tube at the time of test. Once locked, the container is intended to support the test protocol and then be discarded by the appropriate means, as directed by the manufacturer. Re-opening of the lid by the user is inhibited by a mechanical fastening mechanism which is concealed against disengagement and may not be re-opened without damage to the tube, whereby the tube may be understood to be permanently closed.

In another embodiment, a sample tube is provided which permits a lyosphere (substantially solid pellet) or fluid to be added as part of the kit manufacturing process. The sample tube is provided with a first “transport lid” and a second “safety lid”. The transport lid would be utilized to hermetically seal the contents therein during transport. At the time of sample preparation, the transport lid would then be opened, the analyte would be added and the transport lid would then be returned to the closed position. Thereafter, the second safety lid would be placed over the transport lid, to securely lock the contents within the sample tube, thereby minimizing the risk of exposure to the user, test operator or bystanders, again by a mechanical fastening mechanism which is concealed against disengagement and may not be re-opened without damage to the tube, whereby the tube may be understood to be permanently closed.

### BRIEF DESCRIPTION OF THE DRAWINGS

The above-mentioned and other features of this disclosure, and the manner of attaining them, will become more apparent and better understood by reference to the following description of embodiments described herein taken in conjunction with the accompanying drawings, wherein:

FIG. 1 is a perspective view of a first embodiment of a sample tube according to the present disclosure in an opened state;

FIG. 2 is a close-up perspective view of a proximal portion of the tube of FIG. 1;

FIG. 3 is a proximal end (plan) view of the tube of FIG. 1;

FIG. 4 is a distal end (plan) view of the tube of FIG. 1;

FIG. 5 is a cross-sectional side view of the tube of FIG. 1 taken along line 5-5 of FIG. 3;

FIG. 6 is a cross-sectional side view of a proximal portion of the tube of FIG. 1, taken along line 6-6 of FIG. 3;

FIG. 7 is a cross-sectional side view of a proximal portion of the tube of FIG. 1, now in a closed state, taken along line 6-6 of FIG. 3;

FIG. 7A is a portion of the cross-sectional side view of FIG. 7 as bounded by the dashed rectangle 7A in FIG. 7;

FIG. 8 is a perspective view of the tube of FIG. 1 in a closed state;

FIG. 9 is a perspective view of a second embodiment of a sample tube according to the present disclosure in an opened state;

FIG. 10 is a close-up perspective view of a proximal portion of the tube of FIG. 9;

FIG. 11 is another close-up perspective view of a proximal portion of the tube of FIG. 9;

FIG. 12 is a proximal end (plan) view of the tube of FIG. 9;

FIG. 13 is a distal end (plan) view of the tube of FIG. 9;

FIG. 14 is a cross-sectional side view of a proximal portion of the tube of FIG. 9, taken along line 14-14 of FIG. 12;

FIG. 15 is a cross-sectional side view of a proximal portion of the tube of FIG. 9, taken along line 15-15 of FIG. 12;

FIG. 16 is a cross-sectional side view of a proximal portion of the tube of FIG. 9, now in a closed state, taken along lines 14-14 and 15-15 of FIG. 12;

FIG. 16A is a portion of the cross-sectional side view of FIG. 16 as bounded by the dashed rectangle 16A in FIG. 16;

FIG. 17 is a perspective view of the tube of FIG. 9 in a closed state;

FIG. 18 shows the sample tube of FIGS. 1-8 as part of a test kit; and

FIG. 19 shows the sample tube of FIGS. 9-17 as part of a test kit.

### DETAILED DESCRIPTION

It may be appreciated that the present disclosure is not limited in its application to the details of construction and the arrangement of components set forth in the following description or illustrated in the drawings. The invention(s) herein may be capable of other embodiments and of being practiced or being carried out in various ways. Also, it may be appreciated that the phraseology and terminology used herein is for the purpose of description and should not be regarded as limiting as such may be understood by one of skill in the art.

Throughout the description, like reference numerals and letters indicate corresponding structure throughout the drawings/embodiments and, as such, corresponding structure may not be separately discussed as merely being repetitive. Furthermore, any particular feature(s) of a particular exemplary embodiment may be equally applied to any other exemplary embodiment(s) of this specification as suitable. In other words, features between the various exemplary embodiments described herein are interchangeable as suitable, and not exclusive.

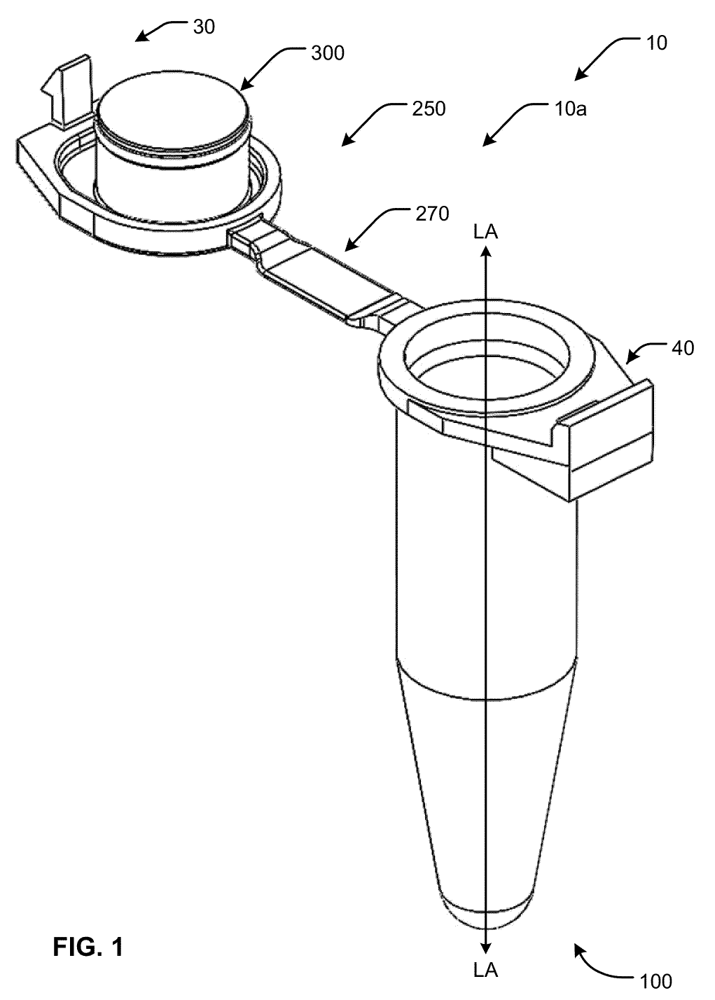

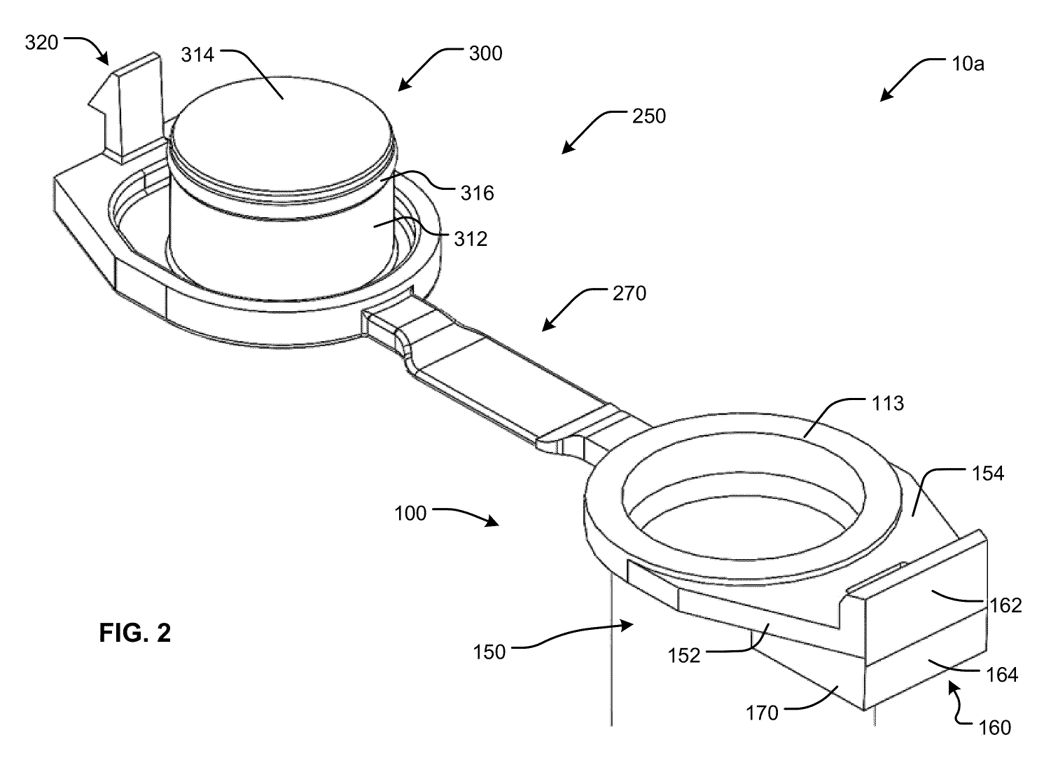

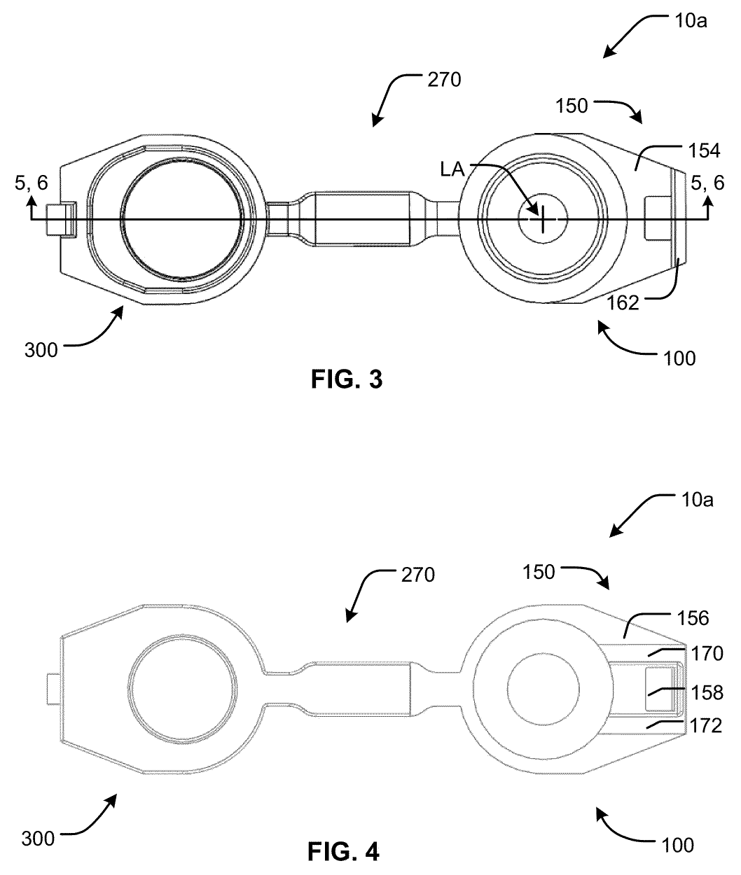

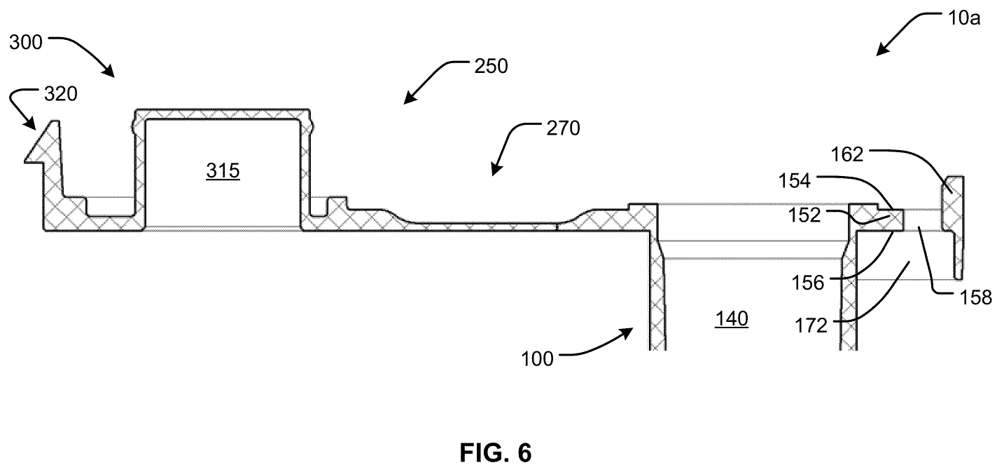

Referring now to FIGS. 1-8, there is shown an exemplary in vitro diagnostic sample tube 10, according to an embodiment of the present disclosure which is further designated as tube 10a. The sample tube 10, which includes sample tubes 10a and 10b, may more particularly be a nucleic acid amplification test (NAAT) tube (which may be abbreviated as a NAAT tube), and more particularly a polymerase chain reaction (PCR) tube (which may be abbreviated as PCR tube).

As shown, the tube 10a comprises a tube body 100 and a tube body cantilevered, hinged (safety) lid 250, which extends laterally from the tube body 100 at a proximal section 102 of the tube body 100, and more particularly a proximal end of the tube body 100. The tube 10a may be formed of plastic, such as via plastic molding, such as injection molding. More particularly, the tube 10a may be formed from thermoplastic using thermoplastic injection molding. As shown, the tube body 100 and the tube body lid 250 are formed as single (monolithic) piece during injection molding. The tube body 100 and the tube body lid 250 may be formed of a thermoplastic composition comprising, essentially consisting of, or consisting of a polyolefin, such as polypropylene (PP) or polyethylene (PE). As molded, the thermoplastic composition is light transmissive (e.g. ultraviolet transparent), as to enable transmittance into the tube 10a, as well as allowing for the reflected light to be measured, such as by a photodetector.

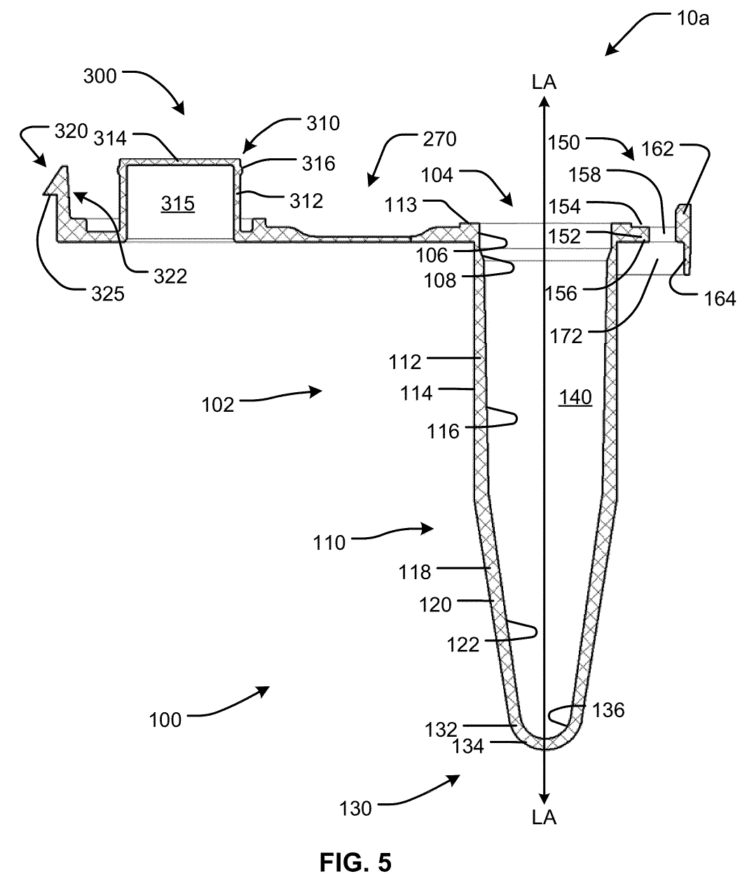

As best shown by FIG. 5, the tube body 100 comprises a proximal (opened end) section 102, an intermediate (or middle) section 110 and a distal (closed end) section 130, which are disposed along a longitudinal axis LA. As shown, the proximal section 102 has a tubular wall 112 which comprises a cylindrical diameter outer surface 114, and a tapering (diameter) inner surface 116, which tapers as the surface 116 extends distally. Distally adjacent the proximal section 102/tubular wall 112, intermediate section 110 comprises a tubular frustoconical wall 118 which has a frustoconical outer surface 120 and a frustoconical inner surface 122. Distally adjacent the intermediate section 110/frustoconical wall 118, the distal (closed end) section 130 comprises a hemi-spherical wall 132 which has a hemi-spherical outer surface 134 and a hemi-spherical inner surface 136.

The tubular body 100 further comprises an in internal cavity 140, which is defined at least in part by inner surface 116, inner surface 122 and inner surface 136.

The proximal section 102 of the tube body 102 includes a proximal end opening 104, which provides both an entrance opening to place test items in the cavity 140. For the tube 10a, the volume of the cavity 140 may be in a range of 0.1 ml to 0.5 ml (milliliters), and more particularly 0.2 ml to 0.5 ml (milliliters). However, for other applications, inclusive of the PCR tube 10a, the sample tube 10 may have a volume in a range of 0.1 ml to 500 ml (milliliters), and more particularly 0.1 ml to 250 ml, and even more particularly 0.1 ml to 100 ml.

The tubular wall 112 of the proximal section 102 further comprises a cylindrical entrance inner surface 106, which defines proximal end opening 104 at the proximal end of the tube body 100, and which is provided in part by an annular (planar) lip 113 at the proximal end of the tube body 100. Distally adjacent the inner surface 106, the inner surface of the proximal section 102 comprises another tapering (diameter) inner surface 108, which is adjacent distal tapering inner surface 116. As shown, tapering inner surface 108 has a taper angle in a range of 15-35 degrees, while tapering inner surface 116 has a taper angle of less than 2-10 degrees. As such, tapering inner surface 108 has a greater taper angle than tapering inner surface 116.

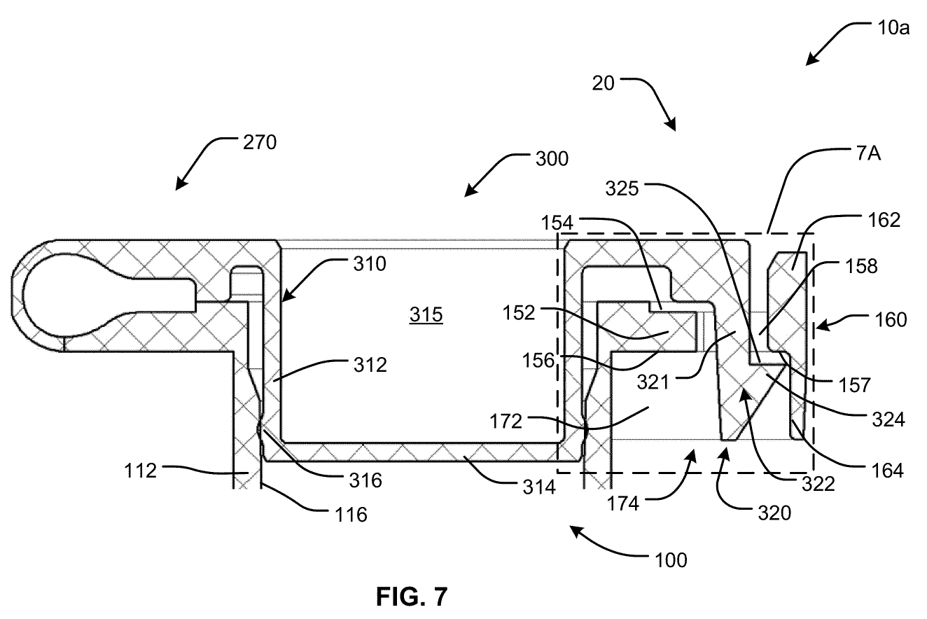

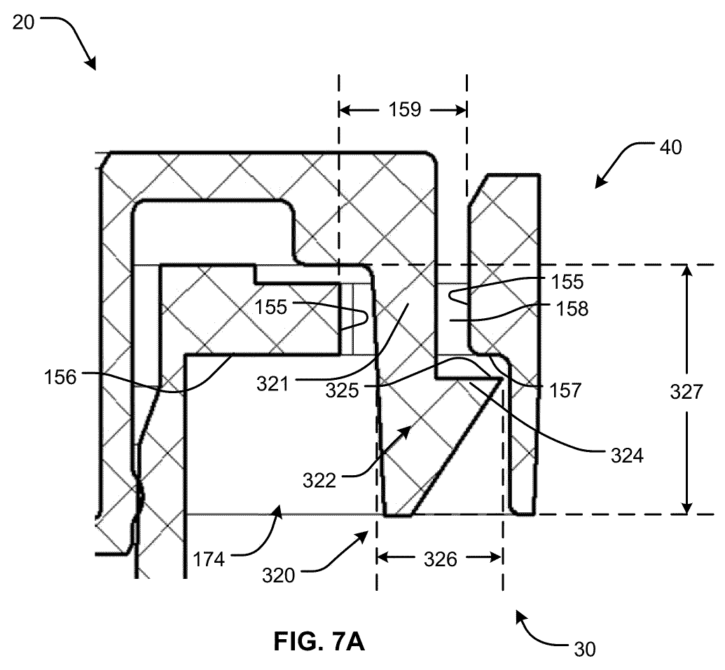

As shown, tube body lid 250 comprises lid cap 300 and a hinge 270. Lid cap 300 comprises a plug 310, which is configured to fit within cavity 140, and form an interference fit seal with the inner surface 116 of tubular wall 112 of the tube body 100, as shown in FIG. 7, to hermetically seal cavity 140 when plug 310 is disposed within cavity 140.

As shown, plug 310 comprises a tubular wall 312 and a circular (planar) distal end wall 314 which define a cylindrical recess 315 in the plug 310. During use of the tube 10a, the recess 315 may be occupied by testing apparatus, such as a stylus to hold/handle the tube 10, a temperature sensing device to sense temperature of the contents of the tube 10 and/or a heater to heat the contents of the tube 10 and reduce condensation during thermal cycling.

The tubular wall 312 further includes a semi-circular protrusion 316 which extends as an annular ring around the circular perimeter of the tubular wall 312 of the plug 310. When plug 310 is disposed within cavity 140, the protrusion 316 forms a resilient interference fit with the tubular wall 112 of the tube body 100 to hermetically seal cavity 140. Thus, it should be understood that the hermetic seal between the tubular body 100 and the plug 310 is formed vertically, and does not need to be formed horizontally with the top surface of the annular ring 113 at the proximal end of the tube body 100.

An interference fit, which may also be referred to as a friction fit or pressure grip fit, may be understood herein as a seal/connection formed between two components which solely relies upon friction to inhibit separation of the components, for example by one of the components being pressed into the other component such that at least one of the components is compressed (deformed) against one another. Here, for example, the semi-circular protrusion 316 of the plug 310 may be compressed, via elastic deformation, when pushed against the tubular wall 112 of the tubular body when the tube 10a is closed.

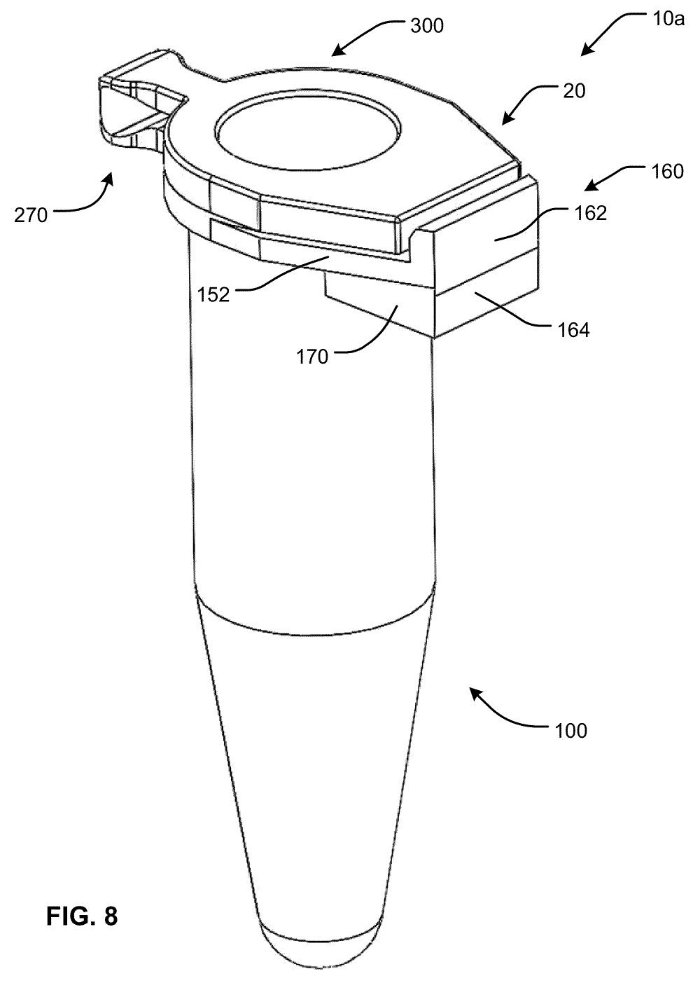

In order to mechanically inhibit the plug 310 from being removed/withdrawn from cavity 140 once the tube 10a is closed, the tube body 100 and the tube body lid 250 cooperate to form a tamper resistant mechanical fastening mechanism 20 (FIGS. 7A, 8), which may be formed by an engagement protrusion 30 (FIGS. 1, 7A) comprising a cantilevered engagement (snap) tab 320 of the lid cap 300, and a engagement protrusion receiver 40 (FIGS. 1, 7A) comprising a (snap) tab catch 157 and surrounding walls of the tubular body 100, which, as explained below, laterally surround and conceal the protrusion 30 within the receiver 40 to inhibit disengagement of the mechanical fastening mechanism 20 once engaged. The mechanical fastening mechanism 20 may provide a purely mechanical connection and, as such, does not rely upon, for example, an adhesive connection, welded connection or other bonded connection.

As shown, the mechanical fastening mechanism 20 comprises a laterally extending platform 150, which extends laterally from the tubular wall 112. More specifically, the laterally extending platform 150 extends transverse to the tubular wall 112, particularly substantially perpendicular (e.g. within 10 degrees of being perpendicular, and inclusive of being perpendicular) to the longitudinal extent of the tubular wall 112/longitudinal axis LA of the tubular body 100.

As shown, laterally extending platform 150 comprises a laterally extending cantilevered wall 152, which has an upper planar side 154 and a lower planar side 156 which are separated by the thickness of the laterally extending platform/wall 150. As shown, the upper side 152 and a lower side 154 are substantially parallel (e.g. within 10 degrees of being parallel, and inclusive of being parallel) to one another. Also as shown, the laterally extending cantilevered wall 152 narrows in width as it extends laterally away from the tubular wall 112.

At a lateral end of the laterally extending cantilevered wall 152, laterally extending platform 150 further comprises a transverse end wall 160. More specifically, the transverse end wall 160 extends transverse to the longitudinal extent of the laterally extending cantilevered wall 152, particularly substantially perpendicular (e.g. within 10 degrees of being perpendicular, and inclusive of being perpendicular) to the laterally extending cantilevered wall 152.

As shown, transverse end wall 160 comprises an upper transverse end wall 162 and a lower transverse end wall 164, which are substantially parallel (e.g. within 10 degrees of being parallel, and inclusive of being parallel) to one another.

As shown, upper transverse end wall 162 and lower transverse end wall 164 extend in opposite directions relative to one another. Upper transverse end wall 162 extends transverse from, and relative to, the laterally extending wall upper side 154, particularly substantially perpendicular (e.g. within 10 degrees of being perpendicular, and inclusive of being perpendicular) to the laterally extending wall upper side 154. Lower transverse end wall 164 extends transverse from, and relative to, the laterally extending wall lower side 156, particularly substantially perpendicular (e.g. within 10 degrees of being perpendicular, and inclusive of being perpendicular) to the laterally extending wall lower side 156.

As shown, the upper transverse end wall 162 and lower transverse end wall 164 form a “T-shaped” wall structure with laterally extending cantilevered wall 152, and each of the upper transverse end wall 162 and lower transverse end wall 164 individually form an “L-shaped” wall structure with laterally extending wall 152,

Laterally extending platform 150 further comprises two spaced reinforcing/brace walls 170, 172 which reinforce/brace the laterally extending cantilevered wall 152 against deformation. As shown the reinforcing/brace walls 170, 172 underlie the laterally extending cantilevered wall 152, which extend along the longitudinal extent of the laterally extending cantilevered wall 152.

As shown, the reinforcing/brace walls 170, 172 extend from the tubular wall 112 to the lower transverse end wall 164. As shown, the reinforcing/brace walls 170, 172 extend transverse from the tubular body 112, the lower side 156 of the laterally extending cantilevered wall 152 and the lower transverse end wall 164, particularly substantially perpendicular (e.g. within 10 degrees of being perpendicular, and inclusive of being perpendicular) to the tubular body 112, the lower side 156 of the laterally extending cantilevered wall 152 and the lower transverse end wall 164. As shown, the reinforcing/brace walls 170, 172 from the tubular wall 112 to the lower transverse end wall 164 are substantially parallel (e.g. within 10 degrees of being parallel, and inclusive of being parallel) to one another.

The laterally extending cantilevered wall 152 further comprises a polygonal (rectangular) window thru-opening 158 which extends through the thickness of the of the wall 152 from the upper side 154 to the lower side 156.

Turning to the tube body lid 250, the lid cap 300 further comprises a cantilevered engagement tab 320, which comprises a cantilevered arm 321 having a pointed (angled/tapering), barbed lead-in engagement (distal) end portion 322 and a hook 324 which is configured to extend through thru-opening 158 in laterally extending wall 152, and hook the lower side 156 of laterally extending wall 152 with laterally extending engagement surface 325 to fasten the mechanical fastening mechanism 20 to hold the tube body lid 300 and the tube body 100 in a closed position. As such, the laterally extending platform 150, and more particularly the laterally extending cantilevered wall 152 provides a catch 157 for the hook 324 of the cantilevered engagement tab 320. As shown, laterally extending engagement surface 325 is transverse to the longitudinal extend of the arm 321, particularly substantially perpendicular (e.g. within 10 degrees of being perpendicular, and inclusive of being perpendicular) to the longitudinal extent of the arm 321. Moreover, when closed, the laterally extending engagement surface 325 and the lower side 156 of the laterally extending platform/wall 150 are substantially parallel (e.g. within 10 degrees of being parallel, and inclusive of being parallel) to one another. Moreover, the complete longitudinal length 327 of the protrusion 30 (cantilevered length of engagement tab 320), is completely disposed within the receiver 40, particularly completely encompassed/surrounded (360 degrees) by the walls of the receiver 40.

As shown in FIG. 7, the hinged lid 250/hinge 270 is folded 180 degrees such that plug 310 is disposed within cavity 140, and the protrusion 316 forming a resilient interference fit with the tubular wall 112 of the tube body 100 to hermetically seal cavity 140. In such regards, it may be understood that the plug 310 and the engagement tab 320 are formed upside down from their proper insertion direction into the cavity 140 and the thru-opening 158, respectively, and must be inverted/rotated for proper insertion via folding of the hinge 270.

The engagement end portion 322 of cantilevered engagement tab 320 extends through thru-opening 158 in laterally extending wall 152 such that the hook 324 engages with the lower side 156 of laterally extending wall 152 to fasten the hook 324 and catch 157 of the mechanical fastening mechanism 20, to mechanically hold the tube body lid 300 and the tube body 100 in a closed position with a positive mechanical engagement. A positive mechanical engagement, which also may be referred to as a mechanical interference, may be understood herein as a connection formed between the components which does not rely solely on friction to inhibit separation of the components and which includes a mechanical interlock to inhibit separation of the components (e.g. overlapping surfaces).

As shown, in addition to supporting laterally extending wall 152 against deformation, the lower transverse end wall 164 and reinforcing/brace walls 170, 172, along with laterally extending wall 152 form a box structure/360 degree enclosed recess 174 around engagement end portion 322/hook 324, in which engagement end portion 322/hook 324 is concealed and laterally surrounded. In such manner, engagement end portion 322/hook 324 is protected against tampering from a lateral contact, which could cause the cantilevered arm 321 of cantilevered engagement tab 320 to resiliently deform towards the tube body 100 (as it may due during engagement to extend through thru-opening 158), causing the hook 324 to possibly disengage.

Referring now to FIG. 7A, as shown, the engagement end portion 322 of the cantilevered arm 321 may have a cross-sectional engagement end portion dimension 326 which is at least 90%, and more particularly at least 95% of the cross-sectional thru-opening dimension 159 of the thru-opening 158. Even more particularly, the engagement end portion dimension 326 of the engagement end portion 322 of the cantilevered arm 321 may have an engagement end portion dimension 326 which is equal to or greater than the thru-opening dimension 159 of the thru-opening 158. For example, the engagement end portion dimension 326 of the engagement end portion 322 of the cantilevered arm 321 may have an engagement end portion dimension 326 which is 0.1% to 100% greater than, and more particularly 0.5% to 50% greater than, and even more particularly 1% to 20% greater than, the thru-opening dimension 159 of the thru-opening 158.

When the engagement end portion dimension 326 of the engagement end portion 322 of the cantilevered arm 321 is greater than the thru-opening dimension 159 of the thru-opening 158, the engagement end portion 322 and/or the thru-opening 158 elastically deform when the engagement end portion 322 is passing through the thru-opening 158 to enable fastening and engage, and thereafter return to shape. Hence, once engaged, the cantilevered arm 321 cannot exit the thru-opening 158 (without contacting the side-wall 155 of the thru-opening 158 provided by the laterally extending wall 152), thus inhibiting the engagement tab 320 from being removed once engaged, particularly without damage to the tube 10a (e.g. breakage of the engagement end portion 322), whereby the tube may be understood to be permanently closed.

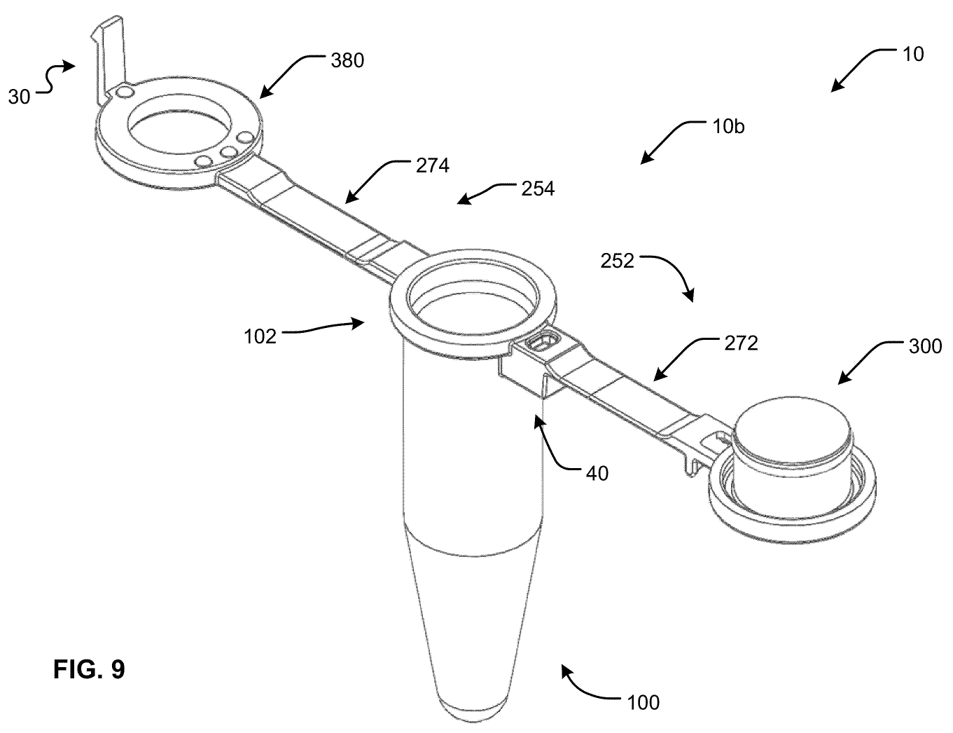

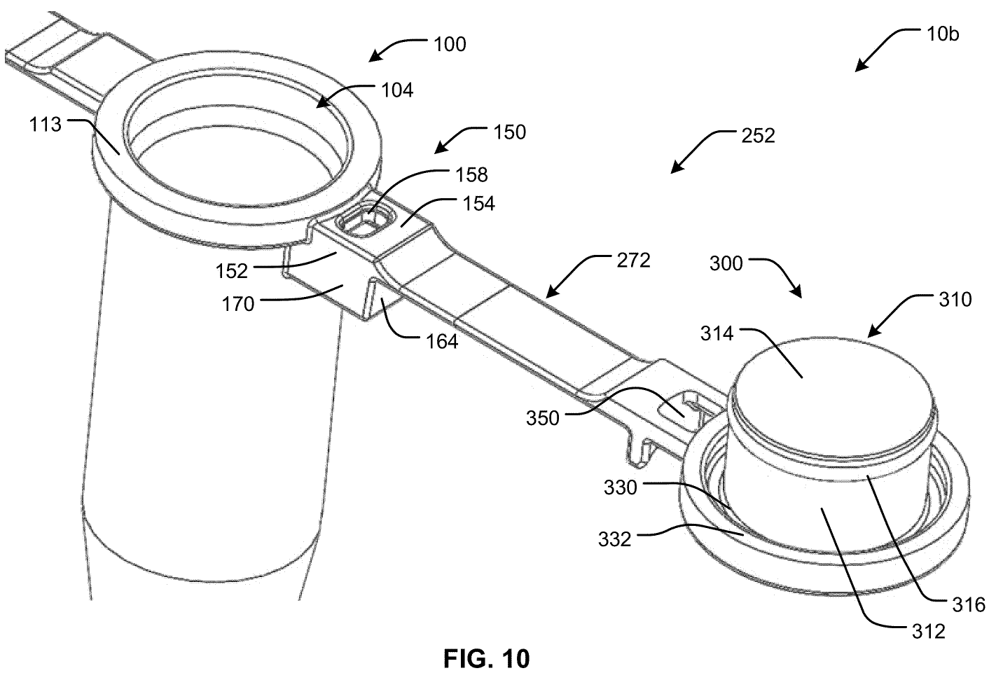

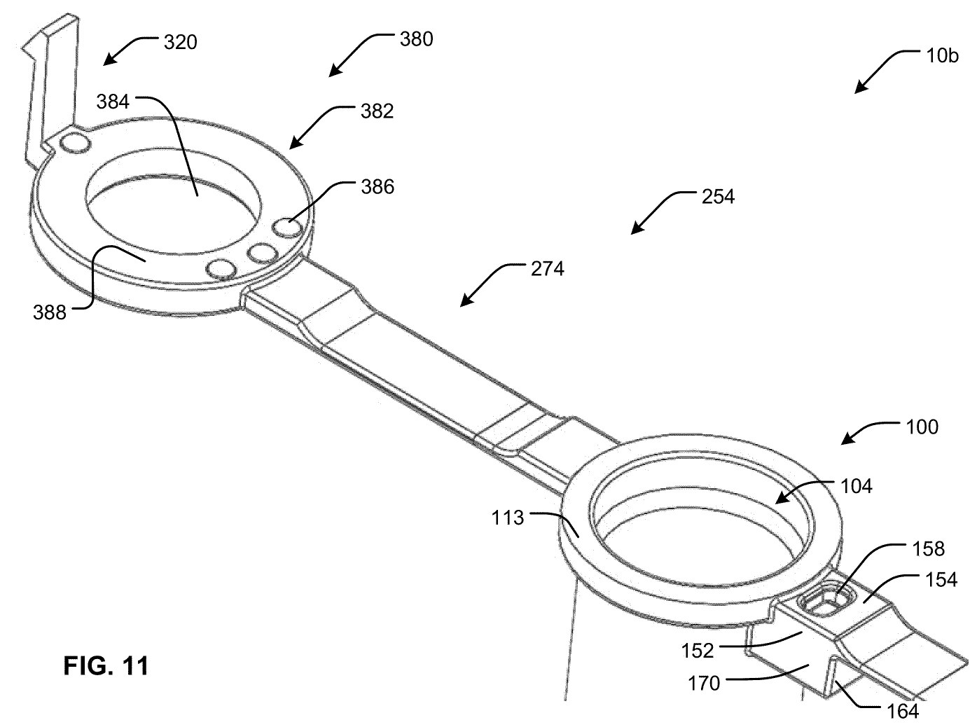

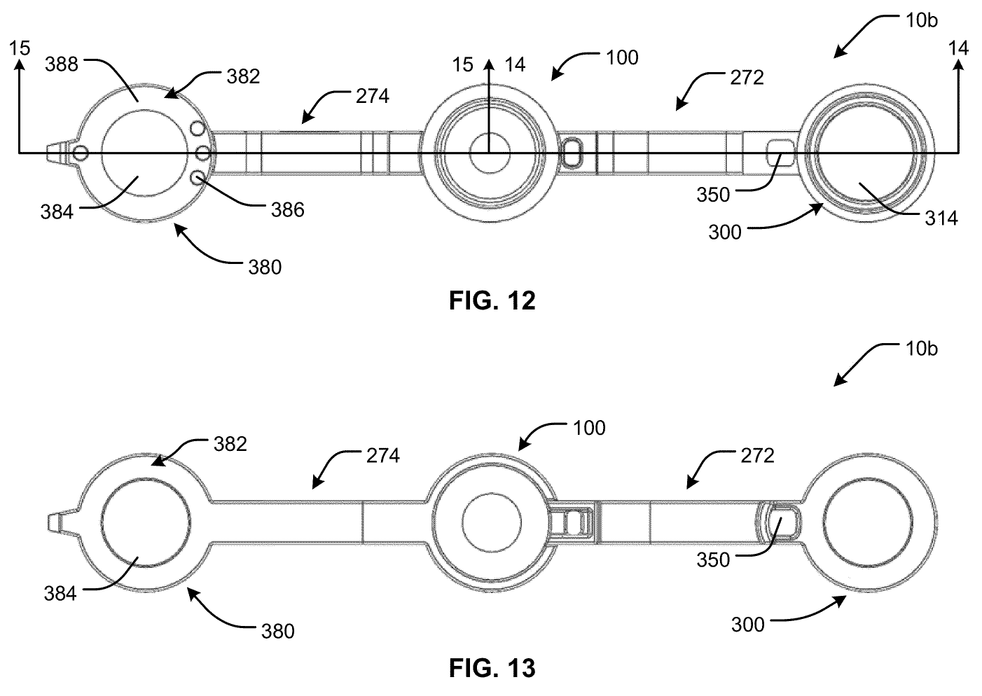

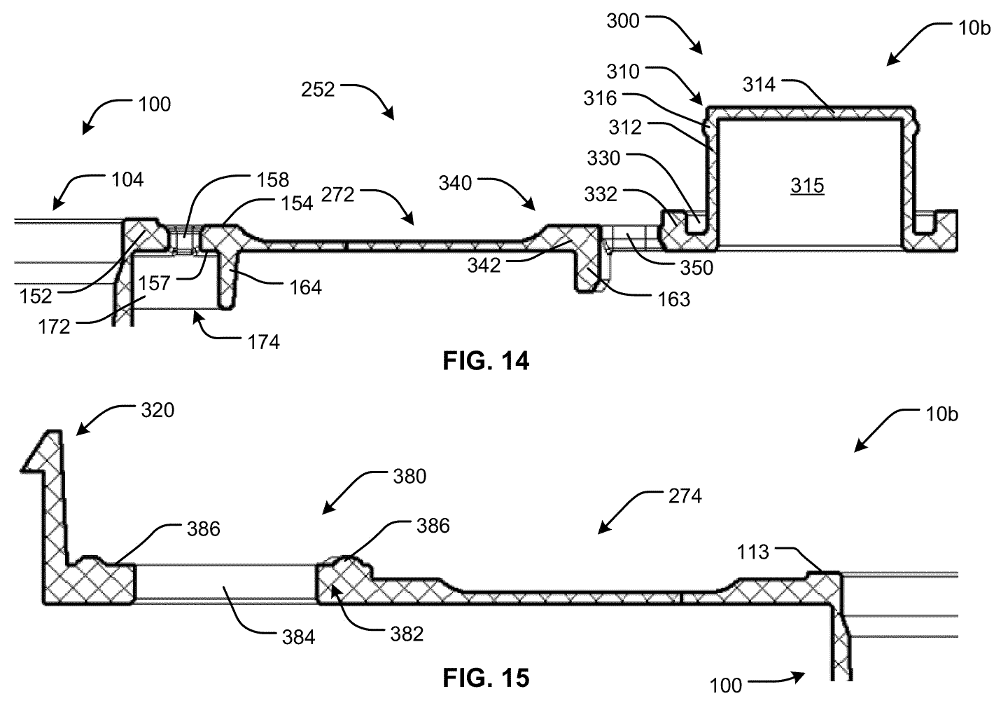

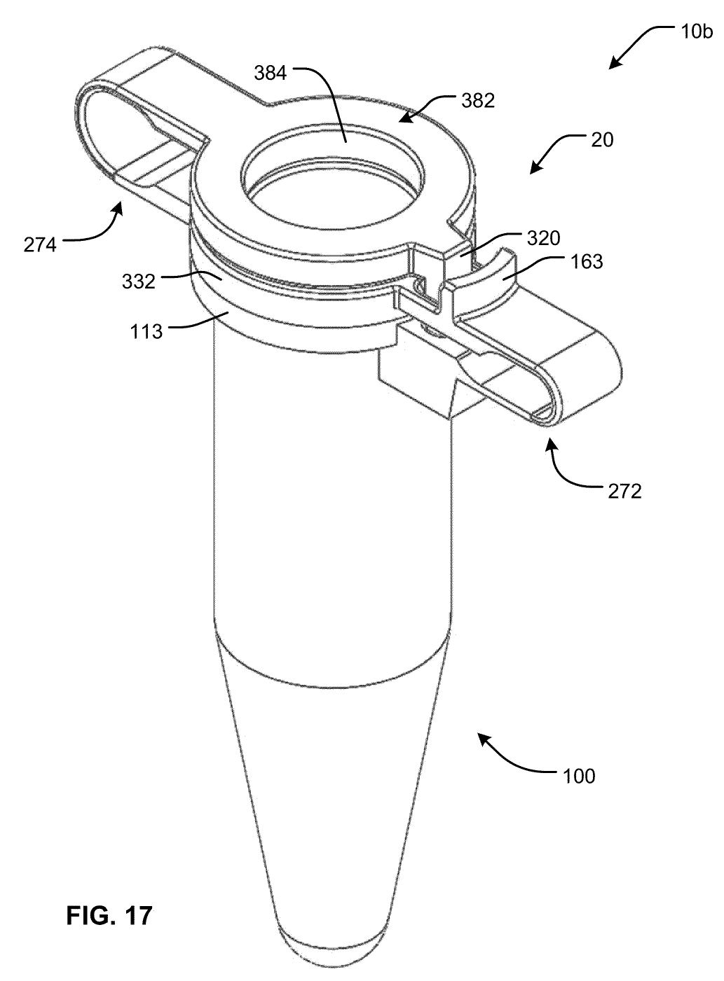

Referring now to FIGS. 9-17, there is shown another sample tube 10 according to another embodiment of the present disclosure, which is further designated as tube 10b. As set forth above, corresponding structure to the earlier embodiment(s), as identified by corresponding reference characters/numerals, may not be discussed, it being understood that such disclosure of the earlier embodiment(s) applies equally to the present embodiment(s) unless otherwise noted.

As shown, the tube 10b comprises a tube body 100, as well as a tube body first cantilevered, hinged (transport) lid 252 and a tube body second cantilevered, hinged (safety) lid 254, which both extend laterally opposite one another from the tube body 100 at a proximal section 102 of the tube body 100, and more particularly a proximal end of the tube body 100. As shown the tube body first hinged lid 252 and the tube body second hinged lid 254 extend in opposite directions such that the tube 10b is “T-shaped”, with the tube body first lid 252 and the tube body second lid 254 extend in opposite directions in a common plane.

As shown, tube body first lid 252 comprises lid cap 300 and hinge 272, which extends laterally from laterally extending platform 150. As shown, upper transverse end wall 162 of the prior embodiment has been eliminated, and replaced with upper transverse end wall 163. As shown, lid cap 300 includes an annular recess 330, which surrounds a portion of the plug 310, and which is defined by an inner side by the plug 310 and an outer side and bottom side by an annular lip 332. As shown, lid cap 300 includes a laterally extending platform 340, between hinge 272 and plug 310, which comprises a laterally extending wall 342 and thru-opening 350 which extend from the annular lip 332.

As shown, tube body second lid 254 comprises a lid cap 380 and hinge 274. As shown, lid cap 380 comprises annular ring 382, which defines a circular thru-opening 384. As shown, the engagement tab 320 is disposed laterally adjacent the annular ring 382.

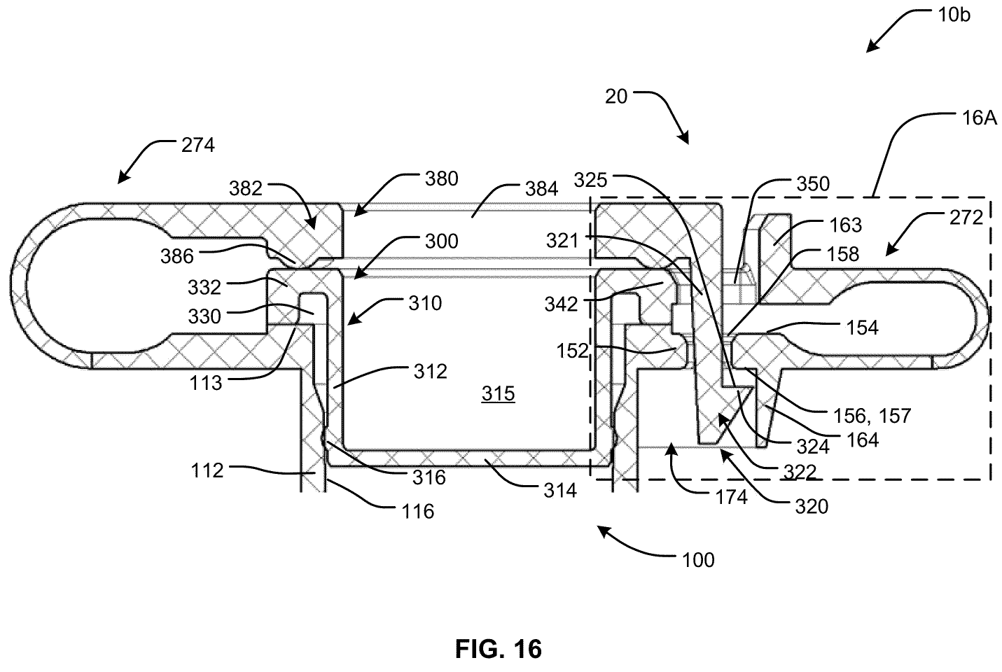

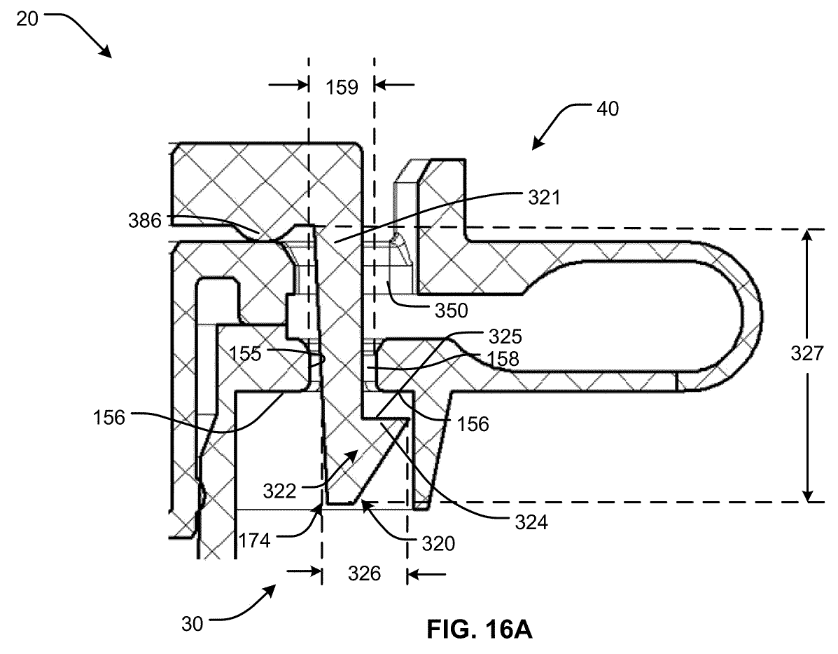

As shown in FIG. 16, the tube body first lid 252/hinge 272 is folded 180 degrees such that plug 310 is disposed within cavity 140, and the protrusion 316 forming a resilient interference fit with the tubular wall 112 of the tube body 100 to hermetically seal cavity 140. As with the prior embodiment, it may be understood that the plug 310 is formed upside down from its proper insertion direction into the cavity 140, and must be inverted/rotated for proper insertion via folding of the hinge 272.

Thereafter, the tube body second lid 254/hinge 274 may be folded 180 degrees such that the engagement tab 320 extends through thru-opening 350 of the tube body first lid 252 and through thru-opening 158 in laterally extending wall 152, and hooks the lower side 156 of laterally extending wall 152, which provides a catch 157 for the hook 324 of the cantilevered engagement tab 320, to mechanically fasten the mechanical fastening mechanism 20 to hold the tube body first lid 252 and tube body second lid 254 and the tube body 100 in a closed position with positive mechanical engagement.

As with the prior embodiment, when the engagement end portion dimension 326 of the engagement end portion 322 of the cantilevered arm 321 is greater than the thru-opening dimension 159 of the thru-opening 158, the engagement end portion 322 and/or the thru-opening 158 elastically deform when the engagement end portion 322 is passing through the thru-opening 158 to enable fastening and engage, and thereafter return to shape. Hence, once engaged, the cantilevered arm 321 cannot exit the thru-opening 158 (without contacting the side-wall 155 of the thru-opening 158 provided by the laterally extending wall 152), thus inhibiting the engagement tab 320 from being removed once engaged, particularly without damage to the tube 10b engagement end portion 322, whereby the tube may be understood to be permanently closed.

As shown, with the present embodiment, the tube body first and second lids 252 and 254 are vertically stacked on each other, with tube body second lid 254 stacked on top of tube body first lid 252. As shown, when both tube body first and second lids 252 and 254 are seated, the cylindrical recess 315 of the plug 310 of the tube body first lid 252 and the cylindrical opening 384 of annular ring 382 of the tube body second lid 254 are aligned along a common longitudinal axis, which is also the center longitudinal axis LA for the tubular body 100. In such manner, the recess 315 and opening 384 may be occupied by testing apparatus, such as a stylus to hold/handle the tube 10, a temperature sensing device to sense temperature of the contents of the tube 10 and/or a heater to heat the contents of the tube 10 and reduce condensation during thermal cycling.

As shown best by FIGS. 16 and 16A, the laterally extending engagement surface 325 of hook 324 and the lower side 156 of the laterally extending wall 152 may be offset by a design tolerance, such that they may not necessarily contact after the tube 10b is closed. In order to adjust this separation gap/design tolerance, the lower side 388 of the annular ring 282 may include at least one protrusion/stand-off 386, the height of which may relatively simply changed in the mold to change or eliminate the separation gap. In which case it may be understood that the cantilevered tab 320 may be in a constant state of tension when engaged. Such protrusion/stand-off 386 may also be incorporated in the first embodiment.

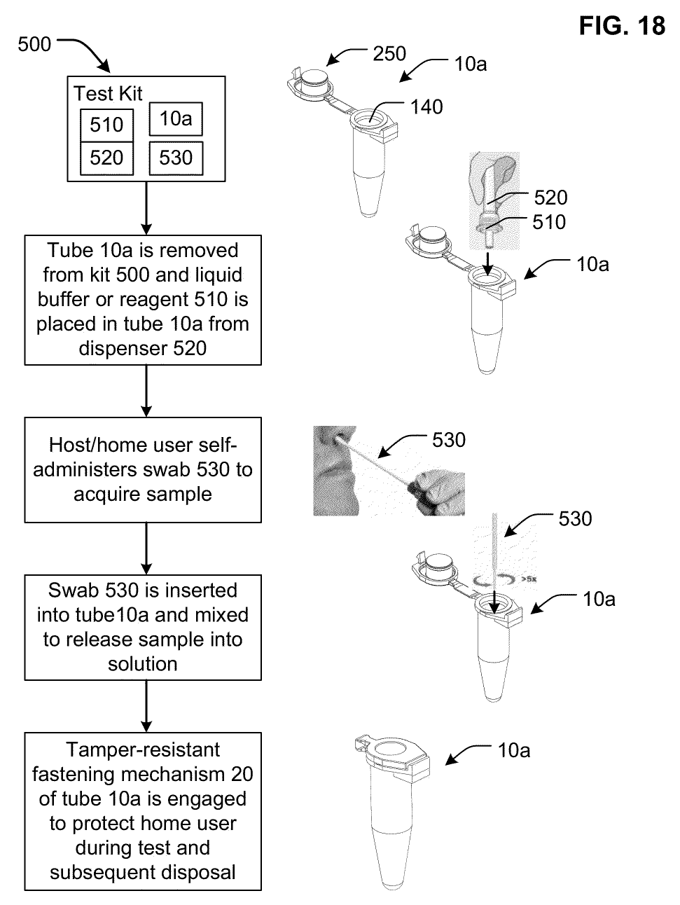

FIG. 18 is a flow diagram of how sample tube 10, and more particularly tube 10a, may be used as part of a viral/pathogen test kit 500 for private home use (e.g. non-commercial/nonclinical), particularly an in vitro diagnostic (IVD) test kit to detect presence of coronavirus disease (COVID-19) disease from severe acute respiratory syndrome coronavirus 2 (SARS-CoV-2).

As shown, the test kit 500 may include tube 10a, a liquid buffer or reagent 510 in a sealed dispenser 520 (e.g. blow-fill-seal container), and a sample collection (e.g. swab) device 530. As shown, once the tube 10a is removed from the kit 500 for use thereof, the liquid buffer or reagent 510 is placed within empty cavity 140 of the tube 10a. Before, during or after the liquid buffer or reagent 510 is placed in the tube 10a, the user of the test kit 500, typically a host and/or home user, may self-administer the sample collection device 530, shown as a nasal swab. Once the liquid buffer or reagent 510 is placed in the tube 10a and the sample is collected by the sample collection device 530, the sample collection device 530 may then be inserted into the cavity 140 of the tube 10a and stirred/mixed with the liquid buffer or reagent 510 disposed within cavity 140 of the tube 10a, after which time the sample collection device 530 may be discarded and the tamper-resistant fastening mechanism 20 of tube 10a is engaged when cavity 140 of tube 10a is closed with lid 250, to protect the home user and others during subsequent testing of the sample and subsequent disposal.

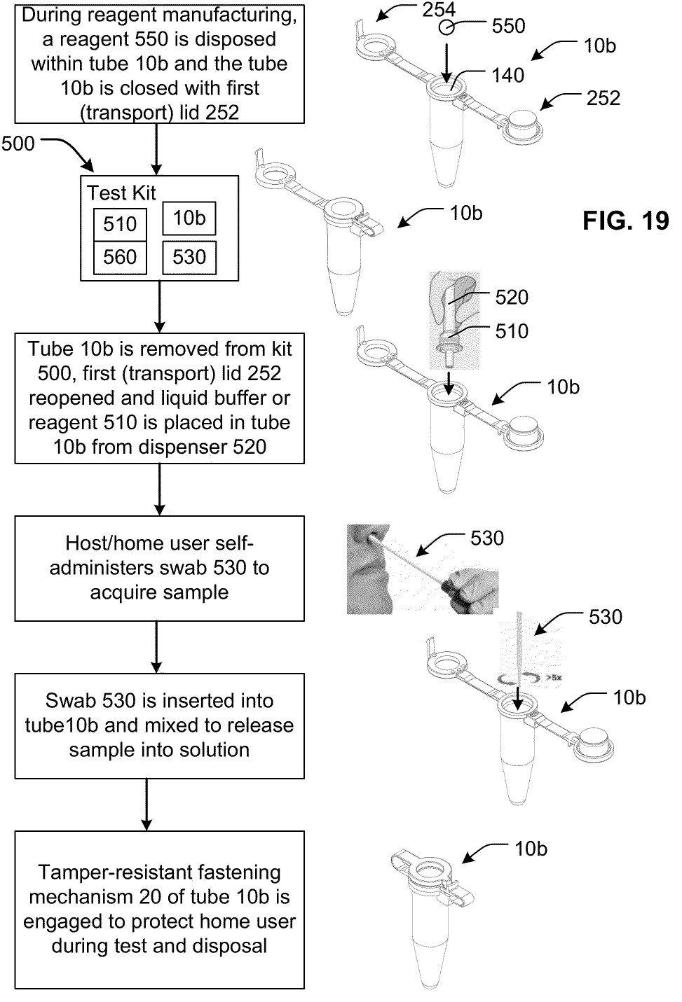

FIG. 19 is a flow diagram of how sample tube 10, and more particularly tube 10b, may be used as part of a viral/pathogen test kit 500 for private home use (e.g. non-commercial/nonclinical), particularly an in vitro diagnostic (IVD) test kit to detect presence of coronavirus disease (COVID-19) disease from severe acute respiratory syndrome coronavirus 2 (SARS-CoV-2).

As, shown, during manufacturing of the test kit 500, a reagent (lyophilized pellet) 550 is placed within the empty cavity 140 of the tube 10b, and the cavity 140 of the tube 10b is then immediately closed with first (transport) lid 252, after which time the tube 10b is disposed in test kit 500. The tube 10b is intended to be filled within an automated filling machine for lyophilized reagent pellets or liquid reagent. Test kit 500 may include tube 10b, a liquid buffer 560 in a sealed dispenser 520, and a sample collection (e.g. swab) device 530. As shown, once the tube 10b is removed from the kit 500 for use thereof, the first (transport) lid 252 is reopened, and the liquid buffer 560 is placed within the cavity 140 of the tube 10b to hydrate and activate the reagents. Before, during or after the liquid buffer 560 is placed in the tube 10b, the user of the test kit 500, typically a host and/or home user, may self-administer the sample collection device 530, shown as a nasal swab. Once the liquid buffer 560 is placed in the tube 10b and the sample is collected by the sample collection device 530, the sample collection device 530 may then be inserted into the cavity 140 of the tube 10b and stirred/mixed with the liquid buffer 560 disposed within cavity 140 of the tube 10b, after which time the sample collection device 530 may be discarded. Thereafter, the cavity 140 is reclosed with first (transport) lid 252, and the tamper-resistant fastening mechanism 20 of tube 10b is engaged with second (safety) lid 254, to protect the home user and others during subsequent testing of the sample and subsequent disposal from the amplicon and reagents.

While a preferred embodiment of the present invention(s) has been described, it should be understood that various changes, adaptations and modifications can be made therein without departing from the spirit of the invention(s) and the scope of the appended claims. The scope of the invention(s) should, therefore, be determined not with reference to the above description, but instead should be determined with reference to the appended claims along with their full scope of equivalents. Furthermore, it should be understood that the appended claims do not necessarily comprise the broadest scope of the invention(s) which the applicant is entitled to claim, or the only manner(s) in which the invention(s) may be claimed, or that all recited features are necessary.

### Listing of Reference Characters

10 tube (10a, 10b)

20 mechanical fastening mechanism

30 protrusion

40 protrusion receiver

100 tube body

102 tube body proximal (opened end) section

104 tube body proximal end opening

106 tube body tubular wall inner surface

108 tube body tubular wall inner surface

110 tube body intermediate section

112 tube body tubular wall

113 tubular body annular lip

114 tube body tubular wall outer surface

116 tube body tubular wall inner surface

118 tube body frustroconical wall

120 tube body frustroconical wall outer surface

122 tube body frustroconical wall inner surface

130 tube body distal (closed end) section

132 tube body hemispherical wall

134 tube body hemispherical wall outer surface

136 tube body hemispherical wall inner surface

140 tube body internal cavity

150 laterally extending platform

152 laterally extending cantilevered wall

154 laterally extending wall upper side

155 thru-opening side-wall

156 laterally extending wall lower side

157 catch

158 thru-opening

159 thru-opening dimension

160 transverse end wall

162 upper transverse end wall

163 upper transverse end wall

164 lower transverse end wall

170 brace wall

172 brace wall

174 recess

250 tube body lid

252 tube body first lid

254 tube body second lid

270 hinge

272 hinge

274 hinge

300 lid cap

310 cap plug

312 cap plug tubular wall

314 cap plug end wall

315 recess

316 cap plug protrusion

320 engagement tab

321 engagement tab arm

322 engagement tab end portion

324 engagement tab hook

325 engagement tab hook engagement surface

326 engagement end portion dimension

327 longitudinal length

330 annular recess

332 annular lip

340 laterally extending platform

342 laterally extending wall

350 thru-opening

380 lid cap

382 annular ring

384 thru-opening

386 protrusion/stand-off

388 annular ring lower side

500 test ki

510 liquid buffer or reagent

520 sealed dispenser

530 sample collection device

550 lyophilized pellet

560 liquid buffer

LA longitudinal axis

What is claimed is:

1. (canceled)

2. A diagnostic sample tube, comprising:
a tube body having a longitudinal axis and a tube cavity to receive a test sample;
a first tube body lid, wherein the first tube body lid comprises a plug configured to fit within the tube cavity and form a seal with the tube body to hermetically seal the tube cavity when the plug is disposed within cavity;
a second tube body lid;
the tube body, the first tube body lid and the second tube body lid are formed as a single piece;
a mechanical fastening mechanism formed with the single piece, the mechanical fastening mechanism comprising an engagement protrusion having a cantilevered engagement tab and an engagement protrusion receiver having a catch;
wherein the cantilevered engagement tab comprises a cantilevered arm having an engagement end portion comprising a hook;
wherein the catch is provided by a laterally extending wall of the tube body which extends transverse to the longitudinal axis of the tube body, wherein the laterally extending wall includes a through-opening;
wherein, when the mechanical fastening mechanism is fastened, the cantilevered engagement tab and the catch fasten the mechanical fastening mechanism with the cantilevered arm occupying the through-opening and the hook extending through the through-opening and engaging the laterally extending wall, which holds the tube body, the first tube body lid and the second tube body lid in a closed position relative to one another with the cavity closed; and
wherein, when the mechanical fastening mechanism is fastened, the engagement protrusion receiver laterally surrounds a longitudinal length of the cantilevered engagement tab including the hook of the cantilevered arm.

3-5. (canceled)

6. The tube of claim 2, wherein:
when the mechanical fastening mechanism is fastened, the cantilevered arm cannot exit the through-opening without the engagement end portion contacting a side-wall of the through-opening.

7. The tube of claim 2, wherein:
the engagement end portion has an engagement end portion dimension;
the through-opening has a through-opening dimension; and
the engagement end portion dimension is greater than the through-opening dimension.

8. The tube of claim 2, wherein:
when the mechanical fastening mechanism is fastened, the portion hook is disposed in a recess of the engagement protrusion receiver.

9. The tube of claim 2, wherein:
the recess of the engagement protrusion receiver is defined at least partially by the laterally extending wall and one or more walls extending extending transverse from the laterally extending wall.

10-14. (canceled)

15. The tube of claim 2, wherein:
the tube cavity has a volume in a range of 0.1 ml to 0.5 ml.

16. The tube of claim 2, wherein:
the tube is a nucleic acid amplification test tube.

17-20. (canceled)

21. The tube of claim 2, wherein:
the tube body second lid comprises the cantilevered engagement tab.

22. (canceled)

23. The tube of claim 2, wherein:
the first tube body first lid comprises a first lid through-opening; and
wherein, when the mechanical fastening mechanism is fastened, the cantilevered arm extends through the first lid through-opening.

24. The tube of claim 2, wherein:
when the mechanical fastening mechanism is fastened, the tube body first lid and the tube body second lid overlap one another.

25. The tube of claim 24, wherein:
when the mechanical fastening mechanism is fastened, the tube body second lid is disposed on top of tube body first lid.

26. The tube of claim 2, wherein:
the first tube body lid comprises a first lid hinge;
the second tube body lid comprises a second lid hinge; and
the tube body, the first tube body lid including the first lid hinge and the second tube body lid including the second lid hinge are formed as the single piece.

27. The tube of claim 2, wherein:
the tube body, the first tube body lid and the second tube body second lid are injection molded and formed of thermoplastic.

28. (canceled)

29. The tube of claim 2, wherein:
the tube is disposed in a kit to test for a presence of SARS-Cov-2 virus in a test sample from a human body.
